(19) United States
(12) Patent Application Publication
(10) Pub. No.: US 2019/0180859 A1
(43) Pub. Date: Jun. 13, 2019

(54) SYSTEM AND METHOD FOR CREATING AN ELECTRONIC DATABASE USING VOICE INTONATION ANALYSIS SCORE CORRELATING TO HUMAN AFFECTIVE STATES

(71) Applicant: BEYOND VERBAL COMMUNICATION LTD., Tel-Aviv (IL)

(72) Inventor: Yoram LEVANON, Ramat Hasharon (IL)

(21) Appl. No.: 16/321,884

(22) PCT Filed: Aug. 2, 2017
(86) PCT No.: PCT/IL2017/050855

§ 371 Date: Jan. 30, 2019

Related U.S. Application Data
(60) Provisional application No. 62/369,770, filed on Aug. 2, 2016.

(51) Int. Cl. G16H 20/70; G16H 50/70; G10L 25/63

(52) U.S. Cl. CPC G16H 20/70; G10L 25/63; G16H 50/70

(57) ABSTRACT

The present invention extends to methods, systems, and devices for creating an electronic database of physiological and psychological states of users and/or groups of users based on acquiring their voice intonation analysis score (VIAS), the method comprises the steps of: applying at least one or more treatment procedures to a user; receiving and analyzing voice input data of the user, based on calculating of average and maximum intensity functions across a plurality of frequencies, said voice input data indicative of speech of the user correlating to said at least one or more treatment procedures; generating and associating a voice intonation analysis score (VIAS) correlating to the physiological and psychological states of said user with said at least one or more treatment procedures that invoked said voice input data; presenting voice intonation analysis score (VIAS) correlating to the physiological and psychological states of said user with said at least one or more treatment procedures that invoked said voice input data; initializing an electronic database to store personal voice intonation analysis score (VIAS) list of one or more treatment procedures to be used as treatment reference indicative of personal unique physiological and psychological states of the user; and updating said electronic database to contain voice intonation analysis score (VIAS) list of one or more treatment procedures to be used as treatment reference indicative of personal unique physiological and psychological states of the user.

### FIELD OF THE INVENTION

The present invention relates generally to the field of measuring and aggregating emotional and physiological responses in human subjects. In particular, the present invention relates to the fields of generating, storing and using semantic networks and databases to correlate physiological and psychological states of users and/or groups of users using their voice intonation data analysis.

### BACKGROUND OF THE INVENTION

The following description includes information that may be useful in understanding the present invention. It is not an admission that any of the information provided herein is prior art or relevant to the presently claimed invention, or that any publication specifically or implicitly referenced is prior art.

Intonation refers to a means for conveying information in speech which is independent of the words and their sounds. It is used to carry a variety of different kinds of information. The interaction of intonation and human affective states is particularly close in many languages. Intonation can derive and use intonation-related phenomena in the voice to make inferences in regards to the information structure of current human affective state of a speaker, including physiological and psychological states, such as excitement, depression, pain and tiredness.

Most of the contemporary human-object interaction systems are deficient in interpreting intonation analysis information derived from human interaction with different physical and virtual objects and suffer from the lack of utilization intelligence by assigning that intonation scale data to a defined human affective state. They are unable to identify truly personal human affective states of a speaker and use this data in providing a solution upon proper actions to improve physiological and/or psychological states. The goal of affective intonation analysis dataset is to fill this gap by detecting and assigning personal physiological and psychological states occurring during human-object interaction and synthesizing physiological and/or psychological responses.

Various systems and methods for indicating emotional attitudes through intonation analysis exist in the art.

U.S. Pat. No. 8,078,470 to Exaudios Technologies Ltd., System for indicating emotional attitudes through intonation analysis and methods thereof, discloses means and method for indicating emotional attitudes of a speaker, either human or animal, according to voice intonation. The invention also discloses a method for advertising, marketing, educating, or lie detecting by indicating emotional attitudes of a speaker and a method of providing remote service by a group comprising at least one observer to at least one speaker. The invention also discloses a system for indicating emotional attitudes of a speaker comprising a glossary of intonations relating intonations to emotions attitudes. The system however does not relate to physiological and psychological states' intonation analysis.

U.S. Pat. No. 7,398,213, to Exaudios Technologies, Method and system for diagnosing pathological phenomenon using a voice signal, relates to a method and system for diagnosing pathological phenomenon using a voice signal. In one embodiment, the existence of at least one pathological phenomena is determined based at least in part upon a calculated average and maximum intensity functions associated with speech from the patient. In another embodiment, the existence of at least one pathological phenomena is determined based at least in part upon the calculated maximum intensity function associated with speech from the patient. The system does not utilize pathological phenomenon analysis into a form of multimodal dataset nor does transform the aggregated data in a proactive helpful application.

Various systems and methods for establishing database associated with emotion analysis are known. Article by Sander Koelstra, “DEAP: A Database for Emotion Analysis using Physiological Signals”, discloses a multimodal dataset for the analysis of human affective states. A method for stimuli selection is proposed using retrieval by affective tags from the last.fm website, video highlight detection and an online assessment tool. An extensive analysis of the participants' ratings during the experiment is presented. Correlates between the EEG signal frequencies and the participants' ratings are investigated. Methods and results are presented for single-trial classification of arousal, valence and like/dislike ratings using the modalities of EEG, peripheral physiological signals and multimedia content analysis. Finally, decision fusion of the classification results from the different modalities is performed. The dataset however does not address to physiological and psychological states on the users based on the intonation analysis.

None of the current technologies and prior art, taken alone or in combination, does not address nor provide a solution for a multimodal dataset using intonation analysis correlating to human affective states, namely generating, storing and using semantic networks and databases to correlate physiological and psychological states of users and/or groups of users based on their intonation analysis.

Therefore, there is a long felt and unmet need for a system and method that overcomes the problems associated with the prior art.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### SUMMARY OF THE INVENTION

It is thus an object of the present invention to provide a method, using a computer processing system, for creating an electronic database of physiological and psychological states of users and/or groups of users based on acquiring their voice intonation analysis score (VIAS), the method comprises the steps of: applying at least one or more treatment procedures to a user; receiving and analyzing voice input data of the user, based on calculating of average and maximum intensity functions across a plurality of frequencies, said voice input data indicative of speech of the user correlating to said at least one or more treatment procedures; generating and associating a voice intonation analysis score (VIAS) correlating to the physiological and psychological states of said user with said at least one or more treatment procedures that invoked said voice input data; presenting voice intonation analysis score (VIAS) correlating to the physiological and psychological states of said user with said at least one or more treatment procedures that invoked said voice input data; initializing an electronic database to store personal voice intonation analysis score (VIAS) list of one or more treatment procedures to be used as treatment reference indicative of personal unique physiological and psychological states of the user; and updating said electronic database to contain voice intonation analysis score (VIAS) list of one or more treatment procedures to be used as treatment reference indicative of personal unique physiological and psychological states of the user.

It is another object of the present invention to provide a system for creating an electronic database of physiological and psychological states of users and/or groups of users based on acquiring their voice intonation analysis score (VIAS), embodied in one or more non-transitory computer-readable media, said system, comprising: at least one processor; and at least one data storage device storing a plurality of instructions and data wherein, upon execution of said instructions by the at least one processor, said instructions cause: apply at least one or more treatment procedures to a user; receive and analyze voice input data of the user, based on calculating of average and maximum intensity functions across a plurality of frequencies, said voice input data indicative of speech of the user correlating to said at least one or more treatment procedures; generate and associate a voice intonation analysis score (VIAS) correlating to the physiological and psychological states of said user with said at least one or more treatment procedures that invoked said voice input data; present voice intonation analysis score (VIAS) correlating to the physiological and psychological states of said user with said at least one or more treatment procedures that invoked said voice input data; initialize an electronic database to store personal voice intonation analysis score (VIAS) list of one or more treatment procedures to be used as treatment reference indicative of personal unique physiological and psychological states of the user; and update said electronic database to contain voice intonation analysis score (VIAS) list of one or more treatment procedures to be used as treatment reference indicative of personal unique physiological and psychological states of the user.

It is another object of the present invention to provide a non-transitory computer-readable medium storing software comprising instructions executable by one or more computers which, upon such execution, cause the one or more computers to perform operations comprising: applying at least one or more treatment procedures to a user; receiving and analyzing voice input data of the user, based on calculating of average and maximum intensity functions across a plurality of frequencies, said voice input data indicative of speech of the user correlating to said at least one or more treatment procedures; generating and associating a voice intonation analysis score (VIAS) correlating to the physiological and psychological states of said user with said at least one or more treatment procedures that invoked said voice input data; presenting voice intonation analysis score (VIAS) correlating to the physiological and psychological states of said user with said at least one or more treatment procedures that invoked said voice input data; initializing an electronic database to store personal voice intonation analysis score (VIAS) list of one or more treatment procedures to be used as treatment reference indicative of personal unique physiological and psychological states of the user; and updating said electronic database to contain voice intonation analysis score (VIAS) list of one or more treatment procedures to be used as treatment reference indicative of personal unique physiological and psychological states of the user.

### BRIEF DESCRIPTION OF THE PREFERRED EMBODIMENTS

The novel features believed to be characteristics of the invention are set forth in the appended claims. The invention itself, however, as well as the preferred mode of use, further objects and advantages thereof, will best be understood by reference to the following detailed description of illustrative embodiment when read in conjunction with the accompanying drawings, wherein:

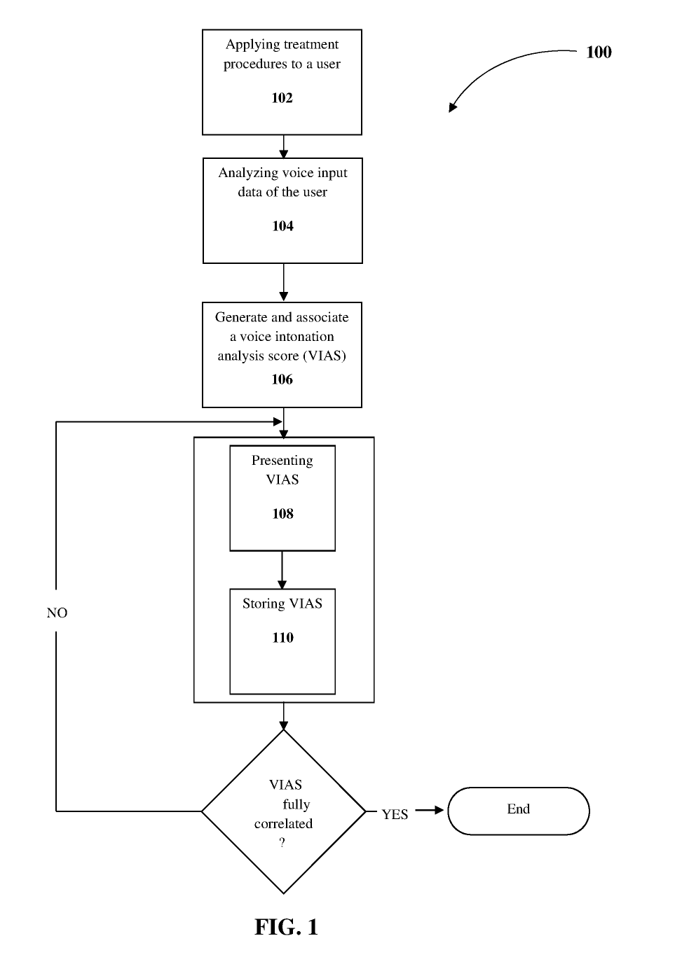

FIG. 1 presents a high level data flow diagram of the method disclosed by the present invention;

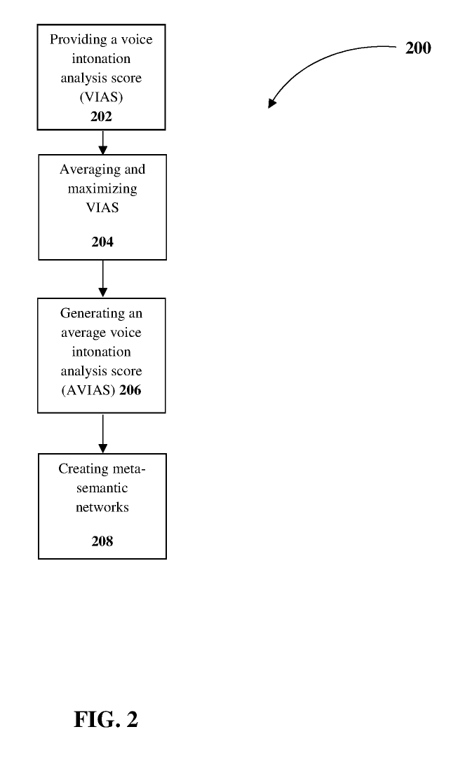

FIG. 2 presents, in topological form, a schematic and generalized presentation of the present invention environment; and

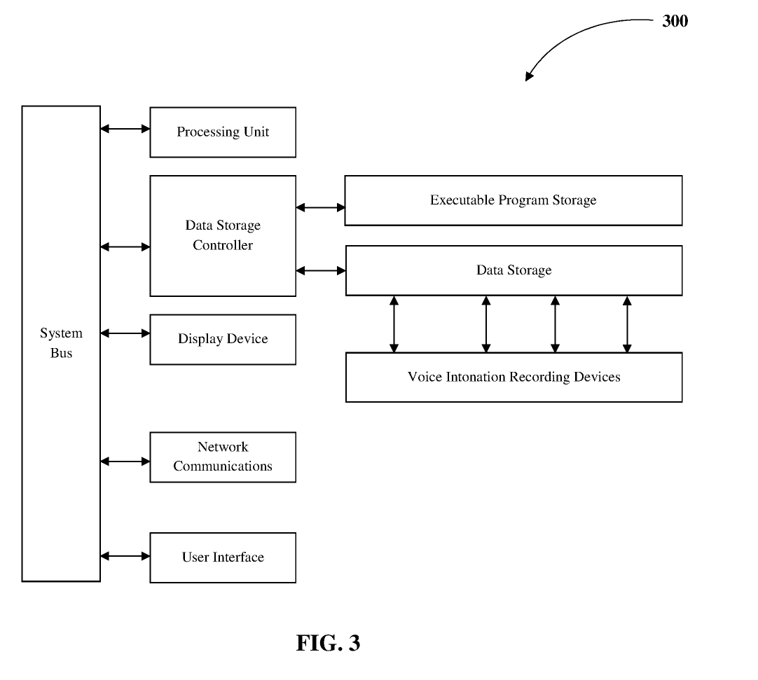

FIG. 3 presents an embodiment of the system disclosed by the present invention.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

While the technology will be described in conjunction with various embodiment(s), it will be understood that they are not intended to limit the present technology to these embodiments. On the contrary, the present technology is intended to cover alternatives, modifications and equivalents, which may be included within the spirit and scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

Unless specifically stated otherwise as apparent from the following discussions, it is appreciated that throughout the present description of embodiments, discussions utilizing terms such as “transmitting”, “calculating”, “processing”, “performing,” “identifying,” “configuring” or the like, refer to the actions and processes of a computer system, or similar electronic computing device. The computer system or similar electronic computing device manipulates and transforms data represented as physical (electronic) quantities within the computer system's registers and memories into other data similarly represented as physical quantities within the computer system memories or registers or other such information storage, transmission, or display devices, including integrated circuits down to and including chip level firmware, assembler, and hardware based micro code.

As will be explained in further detail below, the technology described herein relates to generating, storing and using semantic networks and databases to correlate physiological and psychological states of users and/or groups of users using their voice intonation data analysis.

While the invention is susceptible to various modifications and alternative forms, specific embodiments thereof have been shown by way of example in the drawings and the above detailed description. It should be understood, however, that it is not intended to limit the invention to the particular forms disclosed, but on the contrary, the intention is to cover all modifications, equivalents, and alternatives falling within the spirit and scope of the invention as defined by the appended claims.

The terms “user” used interchangeably in the present invention, refers hereinafter to any party that receives via active and/or passive interaction at least one or more treatment procedures that include but are not limited to prescribed and/or over-the-counter (OTC) medications, medical procedures relating to improving physiological and psychological states of users and/or groups of users, non-medical procedures relating to improving physiological and psychological states of users and/or groups of users, medically-related treatment stimuli and any combinations thereof.

The term “tone” refers in the present invention to a sound characterized by a certain dominant frequencies.

The term “intonation” refers in the present invention to a tone or a set of tones, produced by the vocal chords of a human speaker or an animal.

As a non-limiting example, the implemented creating an electronic database of physiological and psychological states of users and/or groups of users based on acquiring their voice intonation analysis score (VIAS) method can be executed using a computerized process according to the example method 100 illustrated in FIG. 1. As illustrated in FIG. 1, the method 100 can first electronically apply at least one or more treatment procedures to a user 102; receive and analyze voice input data of the user 104, based on calculating of average and maximum intensity functions across a plurality of frequencies, said voice input data indicative of speech of the user correlating to said at least one or more treatment procedures; generate and associate a voice intonation analysis score (VIAS) 106 correlating to the physiological and psychological states of said user with said at least one or more treatment procedures that invoked said voice input data where said voice intonation analysis score (VIAS) receives higher value if said one or more treatment procedures proved to be effective based on the voice input data analysis of the user and where said voice intonation analysis score (VIAS) receives lower value if said one or more treatment procedures proved to be less effective based on the voice input data analysis of the user; present voice intonation analysis score (VIAS) 108 correlating to the physiological and psychological states of said user with said at least one or more treatment procedures that invoked said voice input data; initialize an electronic database to store personal voice intonation analysis score (VIAS) list 110 of one or more treatment procedures to be used as treatment reference indicative of personal unique physiological and psychological states of the user; and if voice intonation analysis score (VIAS) is fully correlated to the physiological and psychological states of said user with said at least one or more treatment procedures that invoked said voice input data, update said electronic database to contain voice intonation analysis score (VIAS) list of one or more treatment procedures to be used as treatment reference indicative of personal unique physiological and psychological states of the user.

As a non-limiting example, the implemented aggregation of voice intonation analysis scores (VIASs) method can be executed using a computerized process according to the example method 200 illustrated in FIG. 2. As illustrated in FIG. 2, the method 200 can first electronically provide voice intonation analysis score (VIAS) 202 correlating to the physiological and psychological states of a group of users with said at least one or more same treatment procedures that invoked said voice input data after different intonation analysis data collected from the group of users correlating to said at least one or more treatment procedures is aggregated together; average and maximize a voice intonation analysis score (VIAS) containing data regarding physiological and psychological states of all the users and/or groups of users 204, based on calculating of average and maximum intensity functions across a plurality of frequencies, said voice input data indicative of speech of the user correlating to said at least one or more treatment procedures and based on applying principal component analysis; generate an average voice intonation analysis score (AVIAS) 206 correlating to the physiological and psychological states of all the users and/or groups of users with said at least one or more treatment procedures that invoked said voice input data; and create one or more meta-semantic networks 208, correlating to said at least one or more treatment procedures that evoke a similar voice intonation analysis score (VIAS) and an average voice intonation analysis score (AVIAS) and linking them together.

Reference is made now to FIG. 3 which graphically illustrates, according to another preferred embodiment of the present invention, an example of computerized system for implementing the invention 300. The systems and methods described herein can be implemented in software or hardware or any combination thereof. The systems and methods described herein can be implemented using one or more computing devices which may or may not be physically or logically separate from each other. Additionally, various aspects of the methods described herein may be combined or merged into other functions.

In some embodiments, the illustrated system elements could be combined into a single hardware device or separated into multiple hardware devices. If multiple hardware devices are used, the hardware devices could be physically located proximate to or remotely from each other.

The methods can be implemented in a computer program product accessible from a computer-usable or computer-readable storage medium that provides program code for use by or in connection with a computer or any instruction execution system. A computer-usable or computer-readable storage medium can be any apparatus that can contain or store the program for use by or in connection with the computer or instruction execution system, apparatus, or device.

A data processing system suitable for storing and/or executing the corresponding program code can include at least one processor coupled directly or indirectly to computerized data storage devices such as memory elements. Input/output (I/O) devices (including but not limited to keyboards, displays, pointing devices, etc.) can be coupled to the system. Network adapters may also be coupled to the system to enable the data processing system to become coupled to other data processing systems or remote printers or storage devices through intervening private or public networks. To provide for interaction with a user, the features can be implemented on a computer with a display device, such as an LCD (liquid crystal display), virtual display, or another type of monitor for displaying information to the user, and a keyboard and an input device, such as a mouse or trackball by which the user can provide input to the computer.

A computer program can be a set of instructions that can be used, directly or indirectly, in a computer. The systems and methods described herein can be implemented using programming languages such as Flash™, JAVA™, C++, C, C#, Visual Basic™ JavaScript™, PHP, XML, HTML, etc., or a combination of programming languages, including compiled or interpreted languages, and can be deployed in any form, including as a stand-alone program or as a module, component, subroutine, or other unit suitable for use in a computing environment. The software can include, but is not limited to, firmware, resident software, microcode, etc. Protocols such as SOAP/HTTP may be used in implementing interfaces between programming modules. The components and functionality described herein may be implemented on any desktop operating system executing in a virtualized or non-virtualized environment, using any programming language suitable for software development, including, but not limited to, different versions of Microsoft Windows™ Apple™ Mac™, iOS™, Android™, Unix™/X-Windows™, Linux™, etc. The system could be implemented using a web application framework, such as Ruby on Rails.

The processing system can be in communication with a computerized data storage system. The data storage system can include a non-relational or relational data store, such as a MySQL™ or other relational database. Other physical and logical database types could be used. The data store may be a database server, such as Microsoft SQL Server™, Oracle™ IBM DB2™, SQLITE™, or any other database software, relational or otherwise. The data store may store the information identifying syntactical tags and any information required to operate on syntactical tags. In some embodiments, the processing system may use object-oriented programming and may store data in objects. In these embodiments, the processing system may use an object-relational mapper (ORM) to store the data objects in a relational database. The systems and methods described herein can be implemented using any number of physical data models. In one example embodiment, an RDBMS can be used. In those embodiments, tables in the RDBMS can include columns that represent coordinates. In the case of environment tracking systems, data representing user events, virtual elements, etc. can be stored in tables in the RDBMS. The tables can have pre-defined relationships between them. The tables can also have adjuncts associated with the coordinates.

Suitable processors for the execution of a program of instructions include, but are not limited to, general and special purpose microprocessors, and the sole processor or one of multiple processors or cores, of any kind of computer. A processor may receive and store instructions and data from a computerized data storage device such as a read-only memory, a random access memory, both, or any combination of the data storage devices described herein. A processor may include any processing circuitry or control circuitry operative to control the operations and performance of an electronic device.

The processor may also include, or be operatively coupled to communicate with, one or more data storage devices for storing data. Such data storage devices can include, as non-limiting examples, magnetic disks (including internal hard disks and removable disks), magneto-optical disks, optical disks, read-only memory, random access memory, and/or flash storage. Storage devices suitable for tangibly embodying computer program instructions and data can also include all forms of non-volatile memory, including, for example, semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The systems, modules, and methods described herein can be implemented using any combination of software or hardware elements. The systems, modules, and methods described herein can be implemented using one or more virtual machines operating alone or in combination with each other. Any applicable virtualization solution can be used for encapsulating a physical computing machine platform into a virtual machine that is executed under the control of virtualization software running on a hardware computing platform or host. The virtual machine can have both virtual system hardware and guest operating system software.

The systems and methods described herein can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network.

Examples of communication networks include, e.g., a LAN, a WAN, and the computers and networks that form the Internet.

One or more embodiments of the invention may be practiced with other computer system configurations, including hand-held devices, microprocessor systems, microprocessor-based or programmable consumer electronics, minicomputers, mainframe computers, etc. The invention may also be practiced in distributed computing environments where tasks are performed by remote processing devices that are linked through a network.

Reference is made now to FIG. 2 which graphically illustrates, according to another preferred embodiment of the present invention, an example of computerized system for implementing the invention 200.

1. A method, using a computer processing system, for creating an electronic database of physiological and psychological states of users and/or groups of users based on acquiring their voice intonation analysis score (VIAS), the method comprises the steps of:
a. applying at least one or more treatment procedures to a user;
b. receiving and analyzing voice input data of the user, based on calculating of average and maximum intensity functions across a plurality of frequencies, said voice input data indicative of speech of the user correlating to said at least one or more treatment procedures;
c. generating and associating a voice intonation analysis score (VIAS) correlating to the physiological and psychological states of said user with said at least one or more treatment procedures that invoked said voice input data;
d. presenting voice intonation analysis score (VIAS) correlating to the physiological and psychological states of said user with said at least one or more treatment procedures that invoked said voice input data;
e. initializing an electronic database to store personal voice intonation analysis score (VIAS) list of one or more treatment procedures to be used as treatment reference indicative of personal unique physiological and psychological states of the user; and
f. updating said electronic database to contain voice intonation analysis score (VIAS) list of one or more treatment procedures to be used as treatment reference indicative of personal unique physiological and psychological states of the user,
wherein said voice intonation analysis score (VIAS) receives higher value if said one or more treatment procedures proved to be effective based on the voice input data analysis of the user; and
wherein said voice intonation analysis score (VIAS) receives lower value if said one or more treatment procedures proved to be less effective based on the voice input data analysis of the user.

2. The method of claim 1, wherein different intonation analysis data collected from different users correlating to said at least one or more treatment procedures is aggregated together.

3. The method of claim 2, wherein the aggregation consists of averaging and maximizing a voice intonation analysis score (VIAS) containing data regarding physiological and psychological states of all the users and/or groups of users.

4. The method of claim 3, wherein the aggregation generates an average voice intonation analysis score (AVIAS) correlating to the physiological and psychological states of all the users and/or groups of users with said at least one or more treatment procedures that invoked said voice input data.

5. The method of claim 2, wherein the aggregation consists of applying principal component analysis.

6. The method of claim 1, wherein said at least one or more treatment procedures that include but are not limited to prescribed and/or over-the-counter (OTC) medications, medical procedures relating to improving physiological and psychological states of users and/or groups of users, non-medical procedures relating to improving physiological and psychological states of users and/or groups of users, medically-related treatment stimuli and any combinations thereof.

7. The method of claim 1, wherein the method further comprising a step of creating one or more meta-semantic networks, correlating to said at least one or more treatment procedures that evoke a similar voice intonation analysis score (VIAS) and linking them together.

8. The method of claim 7, wherein said method further comprising a step of presenting to the user said one or more created meta-semantic networks.

9. A system for creating an electronic database of physiological and psychological states of users and/or groups of users based on acquiring their voice intonation analysis score (VIAS), embodied in one or more non-transitory computer-readable media, said system, comprising:
a. at least one processor; and
b. at least one data storage device storing a plurality of instructions and data wherein, upon execution of said instructions by the at least one processor, said instructions cause:
i. apply at least one or more treatment procedures to a user;
ii. receive and analyze voice input data of the user, based on calculating of average and maximum intensity functions across a plurality of frequencies, said voice input data indicative of speech of the user correlating to said at least one or more treatment procedures;
iii. generate and associate a voice intonation analysis score (VIAS) correlating to the physiological and psychological states of said user with said at least one or more treatment procedures that invoked said voice input data;
iv. present voice intonation analysis score (VIAS) correlating to the physiological and psychological states of said user with said at least one or more treatment procedures that invoked said voice input data;
v. initialize an electronic database to store personal voice intonation analysis score (VIAS) list of one or more treatment procedures to be used as treatment reference indicative of personal unique physiological and psychological states of the user; and
vi. update said electronic database to contain voice intonation analysis score (VIAS) list of one or more treatment procedures to be used as treatment reference indicative of personal unique physiological and psychological states of the user
wherein said voice intonation analysis score (VIAS) receives higher value if said one or more treatment procedures proved to be effective based on the voice input data analysis of the user; and
wherein said voice intonation analysis score (VIAS) receives lower value if said one or more treatment procedures proved to be less effective based on the voice input data analysis of the user.

10. The system of claim 9, wherein different intonation analysis data collected from different users correlating to said at least one or more treatment procedures is aggregated together.

11. The system of claim 10, wherein the aggregation consists of averaging and maximizing a voice intonation analysis score (VIAS) containing data regarding physiological and psychological states of all the users and/or groups of users.

12. The system of claim 11, wherein the aggregation generates an average voice intonation analysis score (AVIAS) correlating to the physiological and psychological states of all the users and/or groups of users with said at least one or more treatment procedures that invoked said voice input data.

13. The system of claim 10, wherein the aggregation consists of applying principal component analysis.

14. The system of claim 9, wherein said at least one or more treatment procedures that include but are not limited to prescribed and/or over-the-counter (OTC) medications, medical procedures relating to improving physiological and psychological states of users and/or groups of users, non-medical procedures relating to improving physiological and psychological states of users and/or groups of users, medically-related treatment stimuli and any combinations thereof.

15. The system of claim 9, wherein said instructions further cause to create one or more meta-semantic networks, correlating to said at least one or more treatment procedures that evoke a similar voice intonation analysis score (VIAS) and linking them together.

16. The system of claim 15, wherein said instructions further cause to present to the user said one or more created meta-semantic networks.

17. A non-transitory computer-readable medium storing software comprising instructions executable by one or more computers which, upon such execution, cause the one or more computers to perform operations comprising:
a. applying at least one or more treatment procedures to a user;
b. receiving and analyzing voice input data of the user, based on calculating of average and maximum intensity functions across a plurality of frequencies, said voice input data indicative of speech of the user correlating to said at least one or more treatment procedures;
c. generating and associating a voice intonation analysis score (VIAS) correlating to the physiological and psychological states of said user with said at least one or more treatment procedures that invoked said voice input data;
d. presenting voice intonation analysis score (VIAS) correlating to the physiological and psychological states of said user with said at least one or more treatment procedures that invoked said voice input data;
e. initializing an electronic database to store personal voice intonation analysis score (VIAS) list of one or more treatment procedures to be used as treatment reference indicative of personal unique physiological and psychological states of the user; and
f. updating said electronic database to contain voice intonation analysis score (VIAS) list of one or more treatment procedures to be used as treatment reference indicative of personal unique physiological and psychological states of the user
wherein said voice intonation analysis score (VIAS) receives higher value if said one or more treatment procedures proved to be effective based on the voice input data analysis of the user; and
wherein said voice intonation analysis score (VIAS) receives lower value if said one or more treatment procedures proved to be less effective based on the voice input data analysis of the user.

18. The non-transitory computer-readable medium of claim 17, wherein different intonation analysis data collected from different users correlating to said at least one or more treatment procedures is aggregated together.

19. The non-transitory computer-readable medium of claim 18, wherein the aggregation consists of averaging and maximizing a voice intonation analysis score (VIAS) containing data regarding physiological and psychological states of all the users and/or groups of users.

20. The non-transitory computer-readable medium of claim 19, wherein the aggregation generates an average voice intonation analysis score (AVIAS) correlating to the physiological and psychological states of all the users and/or groups of users with said at least one or more treatment procedures that invoked said voice input data.

21-24. (canceled)
